(19) United States
(12) Patent Application Publication
(10) Pub. No.: US 2024/0132919 A1
(43) Pub. Date: Apr. 25, 2024

(54) IN SITU METHANOGENIC RECOVERY FROM WASTE PRODUCTS

(71) Applicant: Transworld Technologies Inc., Golden, CO (US)

(72) Inventors: Gary Vanzin, Arvada, CO (US); Joseph Edward Zemetra, Lakewood, CO (US); Roland P. DeBruyn, Highlands Ranch, CO (US); John Deuss, Hamilton (BM)

(21) Appl. No.: 18/547,466

(22) PCT Filed: Feb. 25, 2022
(86) PCT No.: PCT/US2022/070836

§ 371 Date: Aug. 21, 2023

Related U.S. Application Data
(60) Provisional application No. 63/153,732, filed on Feb. 25, 2021.
(60) Provisional application No. 63/229,361, filed on Aug. 4, 2021.

(51) Int. Cl. C12P 5/02; C07C 7/00

(52) U.S. Cl. CPC C12P 5/023; C07C 7/00

(57) ABSTRACT

Exemplary methods of producing methane in a reservoir may include accessing a consortium of microorganisms in a geologic formation. The methods may include delivering an aqueous solution including waste product to the consortium of microorganisms. The methods may include increasing production of gaseous materials by the consortium of microorganisms The methods may include recovering gaseous products from the reservoir. The gaseous products may by characterized by an enriched methane concentration.

### CROSS-REFERENCES TO RELATED APPLICATIONS

This application claims the benefit of, and priority to, U.S. Provisional Application No. 63/153,732, filed 25 Feb. 2021. This application also claims the benefit of, and priority to, U.S. Provisional Application No. 63/229,361, filed 4 Aug. 2021. The contents of both of these applications are hereby incorporated by reference in their entirety for all purposes.

### TECHNICAL FIELD

The present technology relates to methane recovery and production. More specifically, the present technology relates to enhanced methane recovery from biological methane generation in situ utilizing biological waste products.

### BACKGROUND

Increasing world energy demand is creating unprecedented challenges for recovering energy resources, and mitigating the environmental impact of using those resources. Some have argued that the worldwide production rates for oil and domestic natural gas will peak within a decade or less. Once this peak is reached, primary recovery of oil and domestic natural gas will start to decline, as the most easily recoverable energy stocks start to dry up. Historically, old oil fields and carbonaceous formations are abandoned once the easily recoverable materials are extracted.

As worldwide energy prices continue to rise, it may become economically viable to extract additional oil and carbonaceous materials from these formations with conventional drilling and mining techniques. However, a point will be reached where more energy is required to recover the resources than can be gained by the recovery. At that point, traditional recovery mechanisms will become uneconomical, regardless of the price of energy.

Thus, there remains a need for improved methods of recovering oil and other carbonaceous materials from formation environments. There also remains a need for methods of introducing chemical amendments to a geologic formation that will stimulate the biogenic production of methane, which may be used as an alternative source of natural gas for energy production independent of the original reserve of the energy material. These and other needs are addressed by the present technology.

### SUMMARY

Exemplary methods of producing methane in a reservoir may include accessing a consortium of microorganisms in a geologic formation. The methods may include delivering an aqueous solution including waste products to the consortium of microorganisms. The methods may include increasing production of gaseous materials by the consortium of microorganisms. The methods may include recovering gaseous products from the reservoir. The gaseous products may be characterized by an enriched methane concentration.

In some embodiments, the aqueous solution may include less than or about 50 vol. % waste products. The aqueous solution may include produced water extracted from the geologic formation. The gaseous products recovered from the reservoir may be characterized by a concentration of carbon dioxide of less than or about 10 vol. %. The methods may include characterizing the environment of the geologic formation. Characterizing the environment of the geologic formation may include identifying, within the geologic formation environment, one or more of a sulfate concentration, a salinity, a temperature, or a pH. The aqueous solution may include greater than or about 0.1 vol. % glycerol. The methods may include recovering effluent aqueous material at an outlet from the reservoir.

The methods may include monitoring a concentration of carbonaceous waste within the effluent aqueous material. The aqueous solution delivered may be characterized by a pH of less than or about 8. The pH of the aqueous solution may be adjusted by incorporating hydrochloric acid or phosphoric acid.

The methods may include measuring a calcium concentration of an effluent aqueous material recovered from the geologic formation. The methods may include reducing the pH of the aqueous solution with hydrochloric acid when the calcium concentration is measured to be greater than or about 20 mg/L. The methods may include reducing the pH of the aqueous solution with phosphoric acid when the calcium concentration is measured to be less than or about 80 mg/L. The methods may include, when the pH of the aqueous solution is reduced with hydrochloric acid, providing a phosphate compound to the aqueous solution. The aqueous solution delivered into the geologic formation may be characterized by a first concentration of salinity and a first concentration of free fatty acids. A feedstock of the waste products may be adjusted over time to increase carbonaceous material in the aqueous solution. The aqueous solution may be adjusted over time to increase free fatty acids to a second concentration greater than the first concentration of free fatty acids. The aqueous solution may include one or more of yeast extract, inorganic nitrogen, a carboxylate material, a metalloid material, or a metal material.

Some embodiments of the present technology may encompass methods of producing methane in a reservoir. The methods may include accessing a consortium of microorganisms in a geologic formation. The methods may include delivering an aqueous solution incorporating a waste product stream having a carbonaceous waste product at a concentration of greater than or about 10 wt. % to the consortium of microorganisms. The methods may include increasing production of gaseous materials from consumption of the waste products by the consortium of microorganisms. The methods may include recovering gaseous products from the reservoir. The gaseous products may be characterized by a concentration of carbon dioxide of less than or about 40 vol. %.

In some embodiments, the methods may include identifying microorganisms from genus Thermotoga. The methods may include incorporating xylan in the aqueous solution delivered to the consortium of microorganisms. The methods may include separating the carbon dioxide from methane recovered from the reservoir. The methods may include re-injecting the carbon dioxide separated from the methane into the geologic formation.

Some embodiments of the present technology may encompass methods of producing methane in a reservoir. The methods may include accessing a consortium of microorganisms in a geologic formation. The methods may include delivering an aqueous solution incorporating waste products having a glycerol concentration of greater than or about 0.1 vol. % to the consortium of microorganisms. The methods may include increasing production of gaseous materials from consumption of the waste products by the consortium of microorganisms. The methods may include recovering gaseous products from the reservoir. The gaseous products may be characterized by a concentration of methane of greater than or about 51 vol. %. In some embodiments, the methods may include characterizing one or more aspects of the geologic formation selected from a group including temperature, salinity, sulfate concentration, alkalinity, pH, or permeability. The methods may include modifying the one or more aspects of the geologic formation.

Methods of increasing methanogenic activity in a reservoir according to some embodiments of the present technology may include accessing a consortium of microorganisms in a geologic formation that includes a carbonaceous material. The methods may include characterizing the formation environment. The methods may include delivering an aqueous material incorporating glycerol at a concentration of greater than or about 0.1 wt. % to the consortium of microorganisms. The methods may include increasing production of gaseous materials by the consortium of microorganisms. The methods may include recovering methane from the reservoir.

In some embodiments, the methods may include subsequently increasing the concentration of glycerol within the aqueous material to greater than or about 1.0 wt. %. The gaseous materials may include methane, hydrogen, or carbon dioxide. The aqueous material delivered may be characterized by a pH of less than or about 8. The pH of the aqueous material may be adjusted by incorporating hydrochloric acid or phosphoric acid. The methods may include measuring a calcium concentration of an effluent aqueous material recovered from the geologic formation. The methods may include reducing the pH of the aqueous material with hydrochloric acid when the calcium concentration is measured to be greater than or about 20 mg/L. The methods may include reducing the pH of the aqueous material with phosphoric acid when the calcium concentration is measured to be less than or about 80 mg/L. The methods may include, when the pH of the aqueous material is reduced with hydrochloric acid, providing a phosphate compound to the aqueous material.

The methods may include recovering effluent aqueous material at an outlet from the reservoir. The methods may include monitoring a concentration of glycerol within the effluent aqueous material. A glycerol feedstock initially delivered into the geologic formation may be characterized by a first concentration of free glycerol and a first concentration of free fatty acids. The glycerol feedstock may be adjusted over time to reduce free glycerol to a second concentration less than the first concentration. The glycerol feedstock may be adjusted over time to increase free fatty acids to a second concentration greater than the first concentration. The aqueous material may include one or more of yeast extract, a carboxylate material, or a metal material. Characterizing the formation environment may include identifying, within the formation environment, one or more of a sulfate concentration, a salinity, a temperature, or a pH.

Some embodiments of the present technology may encompass methods of increasing methanogenic activity in a reservoir. The methods may include accessing a consortium of microorganisms in a geologic formation that includes a carbonaceous material. The methods may include delivering an aqueous material incorporating waste glycerol at a concentration of greater than or about 1 wt. % to the consortium of microorganisms. The methods may include increasing production of gaseous materials from consumption of the waste glycerol by the consortium of microorganisms. The methods may include recovering methane from the reservoir.

In some embodiments, the methods may include identifying microorganisms from genus Thermotoga. The methods may include incorporating xylan in the aqueous material delivered to the consortium of microorganisms. The methods may include separating carbon dioxide from the methane recovered from the reservoir. The methods may include re-injecting the carbon dioxide separated from the methane into the geologic formation.

Some embodiments of the present technology encompass methods of increasing methanogenic activity in a reservoir. The methods may include accessing a consortium of microorganisms in a geologic formation that includes a carbonaceous material. The methods may include delivering an aqueous material incorporating glycerol at a concentration of greater than or about 1 wt. % to the consortium of microorganisms. The methods may include increasing production of gaseous materials from consumption of the glycerol by the consortium of microorganisms. The methods may include recovering methane from the reservoir.

In some embodiments, the methods may include characterizing one or more aspects of the geologic formation selected from a group including temperature, salinity, sulfate concentration, alkalinity, pH, or permeability. The methods may include modifying the one or more aspects of the geologic formation. The methods may include ramping over time a concentration of glycerol within the aqueous material delivered to the consortium of microorganisms.

Such technology may provide numerous benefits over conventional systems and techniques. For example, by utilizing glycerol or other waste carbonaceous material as a food source for microorganisms, generation of methane may be increased within formation environments. Additionally, the use of waste carbonaceous materials as a source of methane production may allow waste byproducts or materials to be utilized in an environmentally sustainable way. These and other embodiments, along with many of their advantages and features, are described in more detail in conjunction with the below description and attached figures.

### BRIEF DESCRIPTION OF THE DRAWINGS

A further understanding of the nature and advantages of the disclosed technology may be realized by reference to the remaining portions of the specification and the figures.

FIG. 1 is a flowchart illustrating exemplary operations in a method of increasing methanogenic activity from a reservoir according to some embodiments of the present technology.

FIGS. 2A-2D show plots of methane production from the consumption of glycerol according to some embodiments of the present technology.

FIG. 3 is a chart illustrating the synergistic effects of combining supportive waste product streams according to some embodiments of the present technology.

FIG. 4 is a chart illustrating the synergistic effects of combining supportive waste product streams according to some embodiments of the present technology.

FIG. 5 is a chart illustrating antagonistic effects of combining multiple waste product streams according to some embodiments of the present technology.

### DETAILED DESCRIPTION

Biological methane generation is a common source of methane from oil and other reservoirs containing soluble organic compounds. Because microbes use these hydrocarbon materials as a source of carbon, they tend to inhabit areas in reservoirs directly at the interface between these materials and water resident in the formation. In hydrocarbon-bearing environments, the gas present is frequently if not exclusively the result of microbial processing of soluble hydrocarbons, producing methane with specific characteristics that may be nearly identical to gas produced in non-geologic time periods as a result of stimulated methanogenesis. This methanogenic activity may involve accelerating degradation of the carbonaceous material within the reservoir, or may include methane produced by alternative carbon sources. To generate more renewable forms of methane, bioreactors may be used. However, providing sufficient material to support bioreactor activity may be a challenge.

As alternative fuel technology continues to develop, additional carbon sources may be available, and which may be used to produce a sustainable amount of methane. For example, biodiesel is a fuel derived from plants or animals typically produced by chemically reacting lipids, such as animal fat, soybean oil, or some other vegetable oil, with a caustic alcohol solution. This product may provide a ready alternative to petroleum diesel. The popularity of biodiesel has grown significantly over the past two decades, and global production of biodiesel has increased by orders of magnitude in that time. However, with such increased production of biodiesel, the amount of produced byproducts has also increased. Although many of these byproducts may be used in other industries, the significant production of biodiesel has far outpaced demand for the byproducts.

As one example, biodiesel production generates approximately 10 wt. % crude glycerol. Although purified glycerol is used in many products, the extensive amounts of crude waste glycerol resulting from biodiesel production have formed a glut in the byproduct market. Waste glycerol may include any number of impurities, such as salts, alcohols, fatty acids, soaps, and fatty acid methyl esters, and these impurities may be difficult to remove. However, by properly formulating glycerol as a carbon source, crude waste glycerol may be consumed by anaerobic digestion to produce biogas, including methane. A variety of additional carbon-containing waste products similarly may be consumed by anaerobic digestion. By providing a consistent amount of glycerol or other waste products to a microbial consortium, the present technology may produce a sustainable bioreactor for the production of methane.

Additionally, the production of biodiesel includes a cleaning or washing process of the biodiesel products. The washing process removes a number of contaminants from the fuel to improve quality and satisfy fuel stock specification standards, but this washing produces wastewater that may be characterized by significant levels of contaminants and high chemical oxygen demand. Similarly, agricultural processes often consume large amounts of water in treatments and runoff, which may also produce wastewater characterized by increased contaminants rendering the water unsuitable for further use. Any number of other industrial or domestic processes may similarly produce waste including carbonaceous materials and other contaminant materials, which may be termed wastewater, waste products, or carbon-containing waste throughout the present technology. Although water treatment processes may be performed, the processes may be expensive and may still be incapable of sufficient purification. These waste materials, mostly derived from industrial processes, are produced and rapidly oxidized aerobically to carbon dioxide, increasing the global carbon budget without the creation of useable energy. Although attempts have been made to anaerobically degrade these waste products to create methane, many of these waste streams are quite dilute and would require large, expensive capital equipment and long digestion times if used in a conventional, surface anaerobic digester.

These surface anaerobic digesters or ex situ bioreactors may be used to perform microbial digestion of some of these products. However, in addition to the cost issues, these bioreactors may have limited capacity, and may not be environmentally friendly. For example, building bioreactors can be a cost-intensive process, and continually feeding, cleaning, and processing byproducts from ex situ bioreactors may render them cost inefficient. Additionally, byproducts of methane generation may include unusable materials, which may be environmentally taxing. For example, microbial production of methane by consumption of waste carbon sources generates approximately equal amounts of methane and carbon dioxide. The setup of ex situ bioreactors forces this carbon dioxide to be released with the methane. While the methane may be secured and sold, the carbon dioxide is often released, increasing greenhouse gas emissions, and limiting the ability of ex situ reactors to operate as an environmentally friendly source of renewable methane production.

The present technology overcomes the limitations and drawbacks of ex situ reactors by forming an in situ bioreactor within a geologic formation environment, such as a formation environment that may have an additional hydrocarbon material, such as oil, coal, or other carbonaceous materials. By utilizing an in situ bioreactor, carbon sequestration may be enabled, which may reduce or limit the amount of carbon dioxide produced and released. For example, carbon dioxide that is generated in situ may be entrained in the water and form bicarbonate, which may reduce or limit carbon dioxide release from the system. As explained below, bicarbonate may be treated or removed separately, and carbon dioxide generation and release may be limited. Accordingly, gaseous products generated and recovered by the present technology may be characterized by an increased ratio of methane relative to a natural stoichiometric generation of methane with carbon dioxide by methanogenic activity. Similarly, the produced gases may be characterized by a reduced mole fraction of carbon dioxide relative to the stoichiometric production of carbon dioxide as predicted by recovered methane. Consequently, by utilizing waste products discussed throughout the present application as a carbon source, an environmentally friendly, and renewable source of methane may be produced. Although some digestion of the hydrocarbons in the formation may occur, the methane produced may be substantially based on the consumption of delivered carbon materials.

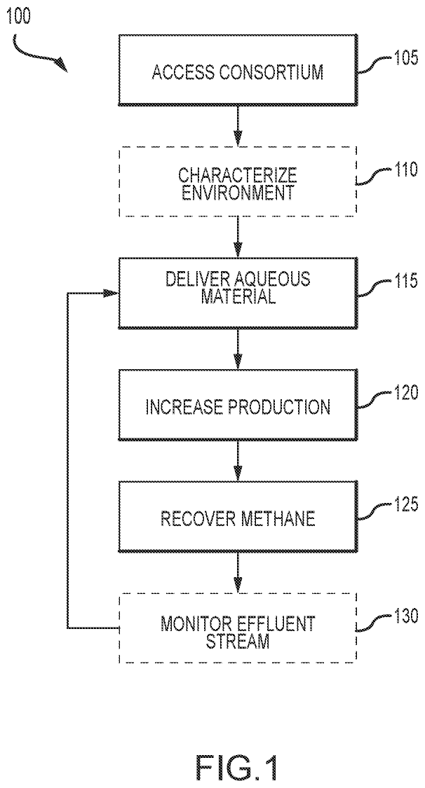

Turning to FIG. 1 is shown a method 100 of increasing methanogenic activity within a reservoir or formation environment, which may be or include production of methane from delivered carbon material for operation as an in situ bioreactor. The method may be designed to stimulate a consortium of microorganisms in the geologic formation to consume waste product components to produce methane and other materials that may be recovered from the geologic formation. The methods performed may stimulate and/or activate a consortium in the formation to start producing the methane, and may increase production of an amount of methane that may be naturally formed within the environment. The methods may further include stopping or decreasing a “rollover” effect such as when the concentration of methane or other metabolic products starts to plateau after a period of monotonically increasing. These and other stimulation effects may be promoted by the materials delivered to the environment according to the method. The method may be performed in a variety of environments, including in situ formation environments as well as ex situ reactors that may be developed, and in which the operations may be performed. However, by performing an in situ process as described, the drawbacks to ex situ production of carbon dioxide may be avoided or limited. Method 100 may include a number of optional operations, which may or may not be specifically associated with some embodiments of methods according to the present technology. For example, many of the operations are described in order to provide a broader scope of the process operations that may be included in some embodiments of the present technology, but are not critical to the technology, or may be performed by alternative methodology as would be readily appreciated.

Method 100 may include accessing a consortium of microorganisms within the geologic formation at operation 105, although as noted above it is to be understood that in some embodiments the methods may be performed in external reactors in which microorganisms may be disposed. The microorganisms may reside in oil, carbonaceous material, formation water, or at an interface between the materials. The geologic formation may be a previously explored oil or hydrocarbon material field in which production may have decreased, or for which increased production may be sought. In other embodiments the geologic formation may be a carbonaceous material-containing subterranean formation, such as a coal deposit, natural gas deposit, carbonaceous shale, or other naturally occurring carbonaceous material, or underground soluble organic compound. In many of these instances, access to the formation can involve utilizing previously mined or drilled access points to the formation. For unexplored formations, accessing the formation may involve digging or drilling through a surface layer to access the underlying site where the microorganisms are located.

Once access to the microorganisms in the formation is available, an analysis of the microorganisms as well as the overall formation environment may be performed in optional operation 110. This may include an in situ analysis of the chemical environment, and/or extracting gases, liquids, or solid substrates from the formation for a remote analysis. Where conditions and characteristics of the formation environment are known, or otherwise accepted, method 100 may be initiated with the delivery of materials to the environment or reactor, whether in situ or ex situ.

When characterization is performed, a number of operations may be performed to

ensure the environment may contain adequate conditions for producing methane, or for accepting glycerol or other carbon waste products for conversion to biogas. Exemplary characterization may include extracting formation samples, which may then be analyzed using spectrophotometry, NMR, HPLC, gas chromatography, mass spectrometry, voltammetry, isotopic analysis, and other chemical instrumentation. The tests may be used to determine the presence and relative concentrations of elements like dissolved carbon, phosphorous, nitrogen, sulfur, magnesium, manganese, iron, calcium, zinc, tungsten, cobalt, and molybdenum, among other elements. The analysis may also be used to measure quantities of polyatomic ions such as PO₂³⁻, PO₃³⁻, and PO₄³⁻, NH₄⁺, NO₂⁻, NO₃⁻, and SO₄²⁻, among other ions. The quantities of vitamins, and other nutrients may also be determined. An analysis of the pH, salinity, oxidation potential, and other chemical characteristics of the formation environment may also be performed.

A biological analysis of the microorganisms may also be conducted to identify methanogenic bacteria, as well as any other beneficial species that may facilitate conversion to methane, including bacteria from the genus Thermotoga, as will be discussed further below. This may include a quantitative analysis of the population size determined by direct cell counting techniques, including the use of microscopy, DNA quantification, phospholipid fatty acid analysis, quantitative PCR, protein analysis, or any other identification mechanism. The identification of the genera and/or species of one or more members of the microorganism consortium by genetic analysis may also be conducted. For example, an analysis of the DNA of the microorganisms may be done where the DNA is optionally cloned into a vector and suitable host cell to amplify the amount of DNA to facilitate detection. In some embodiments, the detecting is of all or part of DNA or ribosomal genes of one or more microorganisms. Alternatively, all or part of another DNA sequence unique to a microorganism may be detected. Detection may be by use of any appropriate means known to the skilled artisan. Non-limiting examples may include 16S Ribosomal DNA metagenomic sequencing; restriction fragment length polymorphism or terminal restriction fragment length polymorphism, polymerase chain reaction, DNA-DNA hybridization, such as with a probe, Southern analysis, or the use of an array, microchip, bead-based array, or the like, denaturing gradient gel electrophoresis, or DNA sequencing, including sequencing of cDNA prepared from RNA as non-limiting examples.

Formation analysis may be performed in embodiments of the present technology to limit negative effects on the microbial community, as well as to ensure efficient conversion of glycerol or other waste products. It is to be understood that although the following discussion will include characteristics of underground environments, the characteristics may be equally applicable to ex situ bioreactors in which production may be sought. For example, microorganisms within a consortium may include both methanogens as well as sulfate reducing bacteria. While methanogens may utilize the delivered waste products to produce methane, sulfate reducing bacteria may cause detrimental effects within the formation environment, and may inhibit the formation of methane. Sulfate reducing bacteria may use hydrogen to reduce sulfate into hydrogen sulfide, which may cause souring and corrosion issues within an oilfield or formation environment. The increase in hydrogen sulfide generation may affect the consortium itself, and may reduce the methanogens relative to other bacteria, which may then reduce methane production and limit additional recovery.

When sulfate is present within the environment, amendments may at certain volumes increase the relative abundance of sulfate reducing bacteria in the formation water. This may then increase the rate of sulfate consumption, which may generate additional hydrogen sulfide gas, limiting production by methanogens. These detrimental effects may be related to the amount of sulfate present in the formation environment. Accordingly, high concentrations of sulfate, such as greater than or about 200 mg/L of formation water, may increase the detrimental effects, and limit enhanced oil recovery. Consequently, in some embodiments the methods may be performed in environments characterized by sulfate concentrations of less than or about 200 mg/L, and may be characterized by sulfate concentrations of less than or about 150 mg/L, less than or about 100 mg/L, less than or about 90 mg/L, less than or about 80 mg/L, less than or about 70 mg/L, less than or about 60 mg/L, less than or about 50 mg/L, less than or about 40 mg/L, less than or about 30 mg/L, less than or about 20 mg/L, less than or about 10 mg/L, or less. Depending on sulfate concentrations within the formation environment, some embodiments of the present technology may employ hydrogen sulfide mitigation techniques, including addition of nitrates or other materials to reduce a sulfate concentration within the formation.

Salinity levels within the formation environment may also be determined. When salinity levels of formation water may be greater than or about 6 vol. %, certain microorganisms in the formation environment may have reduced activity due to the higher salt concentration or brackish water. Additionally, waste glycerol or other waste products may be characterized by a relatively high salt concentration, and which may be an order of magnitude or more greater than the native formation water. As will be explained below, salinity within injected fluid may be controlled by altering the aqueous delivery material, although ensuring the formation environment may have a similar salt concentration to provide compatibility of the delivery material with the formation environment. Accordingly, in some embodiments, salinity, such as sodium concentration, within the formation environment and aqueous material for delivery may be sought or maintained via dilution to less than or about 20,000 mg/L, and may be maintained at less than or about 18,000 mg/L, less than or about 15,000 mg/L, less than or about 12,000 mg/L, less than or about 10,000 mg/L, less than or about 9,000 mg/L, less than or about 8,000 mg/L, less than or about 7,000 mg/L, less than or about 6,000 mg/L, less than or about 5,000 mg/L, less than or about 4,000 mg/L, less than or about 3,000 mg/L, less than or about 2,000 mg/L, or less.

When an underground bioreactor is being used, permeability and/or porosity of the of the formation environment may impact the efficacy of delivery. Glycerol may be characterized by a viscosity that may challenge delivery into the formation environment, as well as through the formation. Additionally, other waste products including semi-solid organic wastes may have a solids concentration or viscosity that can challenge delivery. Waste glycerol or other waste carbon products as may be used in embodiments of the present technology may also contain several percent by weight of ash, which may not be digestible by microorganisms. As this ash or other byproducts build up within the formation, plugging and reduced water movement may occur. Additionally, as the glycerol or other carbon sources are injected into the formation environment, if not sufficiently distributed, microorganisms may preferentially grow near the injection location, which may occlude access through the formation. Depending on the viscosity of the injection fluid, the glycerol or other waste products may also cause pore blocking. As the process is continued over time, permeability may decrease, and thus, in some embodiments the formation environment may be sought or maintained to have a permeability sufficient to support commercial water flow rates, including aqueous materials characterized by increased viscosity, which may be attributable to the incorporation of glycerol, other waste products, or other additives. Increasing permeability or porosity may be performed in any number of ways, including by chemical treatment, delivery of materials to increase fracturing within the formation, or any other process.

Once a suitable environment is identified or developed, method 100 may include delivering an aqueous solution to the formation environment at operation 115. The aqueous solution, which may also include suspended materials in some embodiments, may be delivered by waterflooding to the formation environment. The waterflooding process may involve injection, including pressurized injection, of aqueous materials into the formation environment. In addition to physically displacing oil and other carbonaceous materials, the waterflooding may forcibly deliver the aqueous materials through formation water, as well as permeable surfaces in the formation environment, and which may access the microbial environment. The aqueous solution delivered may include an amount of waste products from any number of operations, including but not limited to biodiesel wastewater or agricultural wastewater, as well as any other wastewater or waste product as described further below. The water or waste product stream may include a carbonaceous material in some embodiments, and may be a carbonaceous waste product. Additionally, aqueous solutions delivered may include an amount of glycerol, which may similarly be termed glycerin, and which may be a part of the wastewater or waste product stream itself, or may be added, such as crude or waste glycerol. The glycerol may include different types of glycerol in some embodiments. For example, biodiesel waste streams may include purified glycerol, crude glycerol, and glycerol bottoms. The present technology may use one or more of these materials in the waste product stream, and may include any combination of the components in some embodiments. The delivered material may be consumed by the microorganisms, which may increase production of gaseous material, such as biogas, at operation 120. The biogas may include methane and hydrogen, and may also include carbon dioxide and other materials. Additionally, the carbon consumption by the microorganisms may also produce liquid materials or carbon-containing liquids. For example, succinic acid may be produced by the microorganisms along with the biogas.

The generated gas and/or liquids may be extracted from the formation environment or otherwise collected at operation 125. For example, recovery from the formation environment may include a combination of water, gas, and other formation materials, such as oil. The gas, oil, and other materials may be separated from the water, which may be used in subsequent delivery. The water may be stored in a holding tank, or directly injected back into the formation environment with additional makeup water, wastewater or waste product streams, and/or other nutrients, allowing the process to be repeated. The water used for the delivery and/or dispersion to the consortium may come from a variety of sources. One source that may be in close proximity to the formation is formation water. Systems and methods for the transport of anaerobic formation water from a subterranean geologic formation may be used to draw formation water from the reservoir, incorporate glycerol and/or additional carbonaceous waste product components as will be described below, and reinject the material back into the formation. Formation water may be anaerobic, and may be characterized as having little or no dissolved oxygen, such as less than or about 4 mg/L, and may have less than or about 2 mg/L, less than or about 0.1 mg/L, or less, as measured at 20° C. and atmospheric pressure. Accordingly, the extraction process may include maintaining anaerobic conditions, or reducing oxygen incorporation prior to re-injection. Any other source of water may also be used in embodiments of the present technology as a source of make-up water, although use of seawater and/or surface water may impact the microorganism consortium, and may contaminate the environment of the reservoir if not properly controlled prior to injection.

Waste products that may be used in some embodiments of the present technology may come from any number of sources that produce wastewater, effluent streams, waste products, or organic wastes having a carbonaceous material content, and which may be consumed by microorganisms within the formation environment. As noted above, waste products or carbonaceous material sources may include biodiesel wastewater or agricultural wastewater, however the present technology may use wastewater or waste streams, which may all be encompassed as waste products, including carbonaceous material from a wide variety of sources. The present technology may utilize any type of waste products including developed waste streams in which waste materials are mixed or solubilized in water, or effluent waste streams produced from one or more other activities.

As non-limiting examples, the present technology may utilize one or more waste product streams including wastes from agriculture, horticulture, aquaculture, forestry, hunting, or fishing; wastes from preparation and processing of meat, fish, or other foods of animal origin, such as effluent streams from slaughterhouses or commercial food production; wastes from fruit, vegetables, vegetation, cereals, edible oils, cocoa, coffee, tea, or tobacco preparation and processing; conserve production, yeast and yeast extract production, molasses preparation and fermentation; wastes from sugar processing; carbon-containing wastes from refineries or other manufacturing plant processes; wastes from dairy products industry; wastes from the baking and confectionary industry; wastes from production or fermentation of alcoholic and non-alcoholic materials or beverages, such as including vinegar production; wastes from wood processing and the production of panels and furniture; wastes from pulp, paper, or cardboard production and processing; wastes from the textiles industry, which may include grease, wax, or other materials; wastes from the manufacture, formulation, supply, or use of basic organic chemicals, and which may or may not include glycerol residues; wastes from the aerobic treatments of wastes, such as a non-composted fraction of municipal or similar wastes; wastes from the anaerobic treatment of wastes, such as liquor from anaerobic treatment of municipal waste or similar wastes; grease or oil mixtures from oil/water separation containing edible oils or fats; garden and park wastes, such as including cemetery waste; or any other waste, residual, or effluent material developed during other material processing.

Waste products according to some embodiments may include semi-solid waste products including wet solids that may remain from filtration or as solids previously entrained, such as from production of food products, for example. As non-limiting examples, vegan or vegetarian-substitute products may be produced from processed feedstocks, including nuts, soy, or plant materials, and residual solids or semi-solid waste materials or wet streams may be incorporated into waste product streams for delivery into the formation environment. Moreover, waste product streams may be developed from renewable biomass products or wastes. As non-limiting examples, renewable biomass products may include material or waste products from planted crops or crop residue, and which may include all annual or perennial agricultural crops from existing agricultural land that may be used as feedstock for renewable fuel, such as grains, oilseeds, or sugarcane, as well as energy crops, such as switchgrass, prairie grass, duckweed, or other planted, ponded, or grown species. Similarly, crop residue may include the biomass left over from the harvesting or processing of planted crops from existing agricultural land or any biomass removed from existing agricultural land that facilitates crop management including biomass removed from such lands in relation to invasive species control or fire management, whether or not the biomass includes any portion of a crop or crop plant. Renewable biomass may include planted trees or tree residue including slash and any woody residue generated during the processing of planted trees from actively managed tree plantations for use in lumber, paper, furniture or other applications. Renewable biomass may include slash and pre-commercial thinnings, biomass obtained from the immediate vicinity of buildings or other areas regularly occupied by people, or of public infrastructure, at risk from wildfire, algae, as well as materials or waste products identified above including animal waste material and animal by-products or separated yard waste or food waste, including recycled cooking or trap grease, dairy and swine manure, landfill, wastewater or wastewater sludge, food waste, green waste, urban landscaping waste, or other organic waste. Additionally, as discussed below, one or more waste streams may be combined to further increase methane production.

Wastewater or aqueous waste products may be characterized by high levels of contaminants and a variable pH based on the process producing the waste product. For example, from acidic wastewater having pH as low as 5, to basic wastewater having pH as high as 11, waste product streams used in embodiments of the present technology may undergo a dilution and/or pH treatment in some embodiments of the present technology. pH treatments will be discussed below, and dilution may facilitate reductions in any number of waste product characteristics, such as chemical oxygen demand, biochemical oxygen demand, suspended solids, and oil and grease. Carbonaceous material content within the waste product streams may be greater than or about 10 wt. % within the aqueous stream, and may be greater than or about 15 wt. %, greater than or about 20 wt. %, greater than or about 25 wt. %, greater than or about 30 wt. %, greater than or about 35 wt. %, greater than or about 40 wt. %, or more.

Additionally, waste product streams used in some embodiments of the present technology may be characterized by a chemical oxygen demand of greater than or about 3,000 mg/L, and may be characterized by a chemical oxygen demand of greater than or about 5,000 mg/L, greater than or about 10,000 mg/L, greater than or about 25,000 mg/L, greater than or about 50,000 mg/L, greater than or about 100,000 mg/L, greater than or about 200,000 mg/L, greater than or about 300,000 mg/L, greater than or about 400,000 mg/L, greater than or about 500,000 mg/L, greater than or about 600,000 mg/L, or greater. Additionally, biochemical oxygen demand may be greater than or about 1,000 mg/L, and may be greater than or about 10,000 mg/L, greater than or about 25,000 mg/L, greater than or about 50,000 mg/L, greater than or about 100,000 mg/L, greater than or about 200,000 mg/L, greater than or about 300,000 mg/L, or greater. Suspended solids may be negligible for some waste product materials, and may be greater than or about 100 mg/L, greater than or about 500 mg/L, greater than or about 1,000 mg/L, greater than or about 2,500 mg/L, greater than or about 5,000 mg/L, greater than or about 10,000 mg/L, greater than or about 20,000 mg/L, greater than or about 30,000 mg/L, or greater. Oil and grease incorporation may be greater than or about 100 mg/L, and may be greater than or about 500 mg/L, greater than or about 1,000 mg/L, greater than or about 2,500 mg/L, greater than or about 5,000 mg/L, greater than or about 10,000 mg/L, greater than or about 20,000 mg/L, greater than or about 30,000 mg/L, greater than or about 40,000 mg/L, or greater.

As will be explained below, sulfate may detrimentally impact the formation environment, and thus in some embodiments waste product streams utilized may be treated or otherwise characterized by a sulfate concentration of less than or about 500 ppm, and may be characterized by a sulfate concentration of less than or about 400 ppm, less than or about 300 ppm, less than or about 200 ppm, less than or about 100 ppm, less than or about 75 ppm, less than or about 50 ppm, less than or about 25 ppm, less than or about 20 ppm, less than or about 15 ppm, less than or about 10 ppm, less than or about 5 ppm, less than or about 2 ppm, or less. Additionally, waste product streams used may be characterized by a conductivity, which may indicate salinity, of less than or about 1,000 μS/cm, and may be characterized by a conductivity of less than or about 800 μS/cm, less than or about 600 μS/cm, less than or about 500 μS/cm, less than or about 400 μS/cm, less than or about 300 μS/cm, less than or about 200 μS/cm, less than or about 100 μS/cm, or less.

By diluting the waste product stream in some embodiments, the concentration of these and other materials described below may be controlled, which may improve consumption ability within the formation environment. For example, formation water, and/or one or more other makeup water sources, may be combined with waste product streams and/or glycerol to produce an aqueous solution of which the waste product, wastewater, or effluent material or stream may be less than or about 50 vol. % of the aqueous solution. In some embodiments, the waste product may be less than or about 45 vol. % of the aqueous solution, and may be less than or about 40 vol. %, less than or about 35 vol. %, less than or about 30 vol. %, less than or about 25 vol. %, less than or about 20 vol. %, less than or about 15 vol. %, less than or about 10 vol. %, less than or about 5 vol. %, less than or about 3 vol. %, less than or about 1 vol. %, or less. This may improve consumption of the materials by microorganisms, which may improve the capability for consumption and allow ramping over time to increase the provided carbonaceous material, and resultant methane production. Additionally, by utilizing a diluted waste product, metal or metalloid precipitates, such as aluminum, boron, barium, iron, zinc, lead, silver, mercury, copper, nickel, chromium, cadmium and tin, which may generate within formation water may be limited.

When utilized or included, glycerol provided into the formation environment may be any type of glycerol including refined glycerol or unrefined glycerol, such as a byproduct from an additional process, and may be glycerol remaining in wastewater. Additionally, in some embodiments additional polyols or hydroxyl-containing materials may be used, including other low-molecular-weight polyols, or polymeric polyols. The polyols may include sugar alcohols, including malitol, sorbitol, xylitol, erythritol, or isomalt, along with any other polyol materials. As noted previously, some embodiments of the present technology may utilize waste glycerol generated from biodiesel production, although waste glycerol from any source may be used according to some embodiments of the present technology. Moreover, in some embodiments a material being incorporated in the aqueous delivery may be any source of carbon, which may be a waste product or byproduct of biofuel production, or any other production process generating waste carbon materials. The waste glycerol may include an amount of free glycerol, as well as methanol, water, and a number of additional chemicals and byproducts. The additional chemicals may include any number of materials, but in some embodiments may include salt, alcohol, soap, fatty acid methyl esters, glycerides, free fatty acids, and ash. The incorporation of these materials may impact consumption by microorganisms, and depending on the concentration, may inhibit or prevent production of methane.

The process to produce methane from waste products and/or glycerol may include a ramping period in which the microorganisms are allowed to grow and become accustomed to the carbonaceous feed stock. Methanogenic bacteria may readily digest free glycerol, methanol and other carbon materials, while having difficulty dealing with soaps, fatty acids, and ash. Consequently, an initiation period for method 100 may include utilizing a first glycerol and/or waste products, which may be characterized by increased free glycerol and/or methanol, or increased carbonaceous waste in the waste product stream, among other beneficial constituent materials. This may be reduced or exchanged over time for less refined glycerol or waste products as the consortium grows and becomes accustomed to digesting glycerol or processed waste product stream carbonaceous material, and the constituent materials in waste glycerol or waste product streams. Additionally, the concentration of glycerol and/or waste product within the stream may be increased over time, which may also increase methane production.

Accordingly, in some embodiments, glycerol addition to the aqueous solution may begin at greater than or about 0.1 wt. % waste glycerol, and may begin at greater than or about 0.2 wt. % waste glycerol, greater than or about 0.5 wt. % waste glycerol, greater than or about 1.0 wt. % waste glycerol, greater than or about 1.5 wt. % waste glycerol, greater than or about 2.0 wt. % waste glycerol, or greater, although the initial amount of glycerol may be maintained below a threshold to ensure consumption by the microorganisms, and to ensure plugging of equipment or the formation environment is limited. Similarly, waste product concentration may begin at a reduced concentration in the aqueous solution, as described above, and may be increased over time in similar percentages as noted.

Determining a threshold for initial delivery, or even delivery over time, may be facilitated by monitoring an effluent stream of aqueous material from the formation environment at optional operation 130. Because the consortium may grow and glycerol-reducing bacteria and/or carbon-consuming bacteria may flourish, the amount of glycerol or carbonaceous material delivery may not remain constant. For example, monitoring an amount of glycerol or carbonaceous waste within the effluent stream may help determine the amount or rate of consumption of glycerol or carbonaceous material by the microorganisms. Consequently, a first glycerol and/or waste carbonaceous material concentration may be provided in the aqueous delivery, and the concentration of glycerol, carbonaceous material, or free fatty acids within the aqueous material subsequently may be increased over time, which may be performed at any ramp rate based on aspects of the environment. As the bioreactor continues to produce and grow in the ability to consume glycerol or carbonaceous material, the concentration of any of these materials may be increased to greater than or about 2 wt. %, and may be increased to greater than or about 5 wt. %, greater than or about 7 wt. %, greater than or about 10 wt. %, greater than or about 12 wt. %, greater than or about 14 wt. %, greater than or about 16 wt. %, greater than or about 18 wt. %, greater than or about 20 wt. %, greater than or about 22 wt. %, greater than or about 24 wt. %, greater than or about 26 wt. %, greater than or about 28 wt. %, greater than or about 30 wt. %, greater than or about 32 wt. %, greater than or about 34 wt. %, greater than or about 36 wt. %, greater than or about 38 wt. %, greater than or about 40 wt. %, or more, although the amount of glycerol or any other viscous or semi-solid materials may be maintained below a threshold that may cause plugging within the formation, such as a concentration of less than or about 80 wt. %, less than or about 60 wt. %, or less.

It is to be understood that delivery concentration may be adjusted based on a volume of dilution material resident in the formation environment. For example, within the formation environment, a concentration of glycerol or waste product carbonaceous material may be diluted subsequent to injection, and may be diluted by a factor of two or more from any of the numbers noted above, and may be diluted to any extent previously noted. As one non-limiting example, if glycerol is delivered at an incorporation percentage of 2 wt. %, dilution by a factor of two may produce an incorporation of 1 wt. % within the formation environment. In some embodiments the dilution of any previously noted concentration may be a dilution by greater than or about a factor of 2, greater than or about a factor of 3, greater than or about a factor of 5, greater than or about a factor of 10, greater than or about a factor of 15, greater than or about a factor of 20, greater than or about a factor of 25, greater than or about a factor of 30, greater than or about a factor of 35, greater than or about a factor of 40, greater than or about a factor of 45, greater than or about a factor of 50, or more. In some embodiments, measuring concentration within the formation environment may be performed by accessing the formation in one or more locations and removing formation water, followed by identifying a concentration of glycerol or waste product carbonaceous material within the removed portion.

Determining the rate of increase for glycerol or waste product stream delivery may be performed based on material concentration at an outlet of the formation environment, which optionally may be monitored during method 100. For example, glycerol or carbonaceous material concentration in the material delivered may be increased as glycerol or carbonaceous waste at the production reduces to less than or about 5.0 wt. %, less than or about 4.0 wt. %, less than or about 3.0 wt. %, less than or about 2.0 wt. %, less than or about 1.0 wt. %, less than or about 0.50 wt. %, less than or about 0.10 wt. %, less than or about 0.050 wt. %, less than or about 0.010 wt. %, or less. Although recovered aqueous material may be reused in subsequent injections, limiting glycerol or carbonaceous waste concentration at the outlet may improve overall conversion and material use.

Similarly to the increase in concentration over time, the purity of the material may be reduced over time, which may allow a more cost-effective waste glycerol or waste product stream to be used. For example, an initial glycerol material may be characterized by a free glycerol concentration within the waste glycerol of greater than or about 20%, and may be characterized by a free glycerol concentration of greater than or about 22%, greater than or about 24%, greater than or about 26%, greater than or about 28%, greater than or about 30%, greater than or about 32%, greater than or about 34%, greater than or about 36%, greater than or about 38%, greater than or about 40%, greater than or about 42%, greater than or about 44%, greater than or about 46%, greater than or about 48%, greater than or about 50%, or higher. Similarly, an initial glycerol material incorporated in the aqueous material may be characterized by a methanol concentration of greater than or about 5%, and may be characterized by a methanol concentration of greater than or about 6%, greater than or about 7%, greater than or about 8%, greater than or about 9%, greater than or about 10%, greater than or about 11%, greater than or about 12%, greater than or about 13%, greater than or about 14%, greater than or about 15%, or higher. Additionally, waste product streams may be characterized by an initial carbonaceous material concentration of greater than or about 20% or more, before utilizing waste product streams characterized by lower or higher concentrations over time, as the microbial community adapts. These concentrations may be achieved by processing or refining the waste glycerol or waste product. Over time, these concentrations may then be reduced to any of the numbers shown, which may reduce a cost of the material.

As noted above, additional materials within the waste glycerol or waste product may inhibit generation of methane in higher concentrations. However, these same materials may be beneficial to the conversion when the microbial community grows and can better adapt to the materials. For example, while soaps, fatty acid methyl esters, and free fatty acids may inhibit methanogenic activity initially, when the consortium has adapted to the materials, these same materials may provide better stoichiometric conversion of carbon material to methane. Accordingly, while remaining below inhibitory thresholds, these materials may help increase methane generation beyond free glycerol or singular waste products alone. The concentrations of these inhibitory materials may be diluted based on the waste glycerol or waste product concentration within the aqueous solution, although maintaining these materials below thresholds within the waste glycerol or waste product stream initially may beneficially allow the microorganisms to increase consumption capability over time. Consequently, initial concentrations of these materials within glycerol or waste products may be reduced or limited initially, while allowing or causing the concentrations to increase over time.

Soaps included within the material may directly solubilize or destroy microorganisms, and hence, soap concentration in waste glycerol or waste product streams at an initial delivery may be maintained below or about 30 wt. %, and may be maintained below or about 28 wt. %, below or about 26 wt. %, below or about 24 wt. %, below or about 22 wt. %, below or about 20 wt. %, below or about 18 wt. %, or less. Over time, as the microorganism population increases and may overcome effects of soaps, these amounts may be allowed to increase towards received waste glycerol or waste product stream amounts without further refinement, and may be allowed to increase to greater than or about any of the numbers stated above.

Free fatty acids included within the material may directly inhibit microbial activity, although at low concentrations, may be an additional food source. Hence, during initial delivery, free fatty acid concentration in waste glycerol or waste product streams may be maintained less than or about 5 wt. %, and may be maintained less than or about 4 wt. %, less than or about 3 wt. %, less than or about 2 wt. %, less than or about 1.5 wt. %, less than or about 1.0 wt. %, less than or about 0.5 wt. %, or less. Fatty acid methyl esters may be one of the main constituents of biodiesel, and may be maintained within waste glycerol to levels of less than or about 30 wt. %, and may be maintained to less than or about 28 wt. %, less than or about 26 wt. %, less than or about 24 wt. %, less than or about 22 wt. %, less than or about 20 wt. %, less than or about 18 wt. %, less than or about 16 wt. %, or less. Over time, again as the microorganism population grows and increases the capability of consuming these materials, the amounts may be allowed to increase towards received waste glycerol or waste product amounts without further refinement, and may be allowed to increase to greater than or about any of the numbers stated above.

Formation water or fluids in which the microorganisms may be found may be characterized by a relatively neutral pH. Delivering aqueous materials at a pH similar to the formation environment may ensure the microorganisms may remain viable. Glycerol and waste product streams may be characterized by a relatively high pH, and may be provided at a pH of greater than or about 11, although as noted above, wastewater may also be characterized by acidic pH. If provided unabated, and especially if the concentration delivered is increased over time, the pH may affect the formation environment, resulting in reduced methane production. Accordingly, in some embodiments, waste glycerol or waste product streams may be modified to reduce or adjust the pH, and ensure the pH of the aqueous material delivered is less than or about pH 8. A slightly higher pH of the injection material may also aid in marginal increases of pH within the formation environment. In slightly acidic environments, this increase may improve activity of the microorganisms, and further increase methanogenic production. When pH reduction is performed, the glycerol or waste product may be modified to reduce the pH with an acid, such as hydrochloric acid or phosphoric acid, as well as with weak organic acids, such as citric acid or acetic acid. If the pH of the material is to be raised, any number of caustic agents may be added to provide a desired pH range.

As will be explained below, addition of nutrients, such as phosphorus, may further increase methane production. Accordingly, reducing pH with phosphoric acid may additionally provide phosphorus to the aqueous solution. However, adding nutrients to the subsurface, such as phosphate compounds or nitrogen compounds, can elicit precipitation reactions. For example, phosphate compounds can be created, including compounds such as hydroxyapatite or struvite, which may hinder water flow within the reservoir along with glycerol or other material distribution. A number of factors may influence these reactions, including temperature, pH, reduction/oxidation potential, as well as the natural concentration within the environment of minerals and metals such as calcium, magnesium, manganese, phosphate, sodium, bicarbonate, carbon dioxide, iron, and others.

Modeling may be developed to analyze geochemical data and environmental characteristics to produce a saturation index, and facilitate a determination of the likelihood of precipitation occurring. In some embodiments, variables utilized in the model may include the temperature within the formation, as well as calcium concentration. As one example, these and other variables may influence an amount of phosphate that may be incorporated within the aqueous material being injected. When a likelihood of precipitation of phosphate compounds may increase, phosphoric acid may not be used to reduce pH of the glycerol or waste product stream, and instead alternative acids such as hydrochloric acid may be used. The likelihood of precipitation may increase at higher temperatures as well as higher calcium concentrations. Accordingly, phosphoric acid may be used with or instead of other acids when the formation temperature is less than or about 80° C., and may be used when the formation temperature is less than or about 75° C., less than or about 70° C., less than or about 65° C., less than or about 60° C., less than or about 55° C., less than or about 50° C., less than or about 45° C., less than or about 40° C., or less. Additionally, phosphoric acid may be used when calcium concentration within the formation environment is less than or about 100 mg/L, and may be used when calcium concentration is less than or about 90 mg/L, less than or about 80 mg/L, less than or about 70 mg/L, less than or about 60 mg/L, less than or about 50 mg/L, less than or about 40 mg/L, less than or about 30 mg/L, less than or about 20 mg/L, or less.

Above any or all of the ranges noted above, a likelihood of precipitation of phosphate materials may increase, and in some embodiments phosphoric acid may not be used to reduce glycerol pH. Instead, hydrochloric acid or some other acid may be used to neutralize the glycerol or waste product streams. Phosphate incorporation may still benefit methane generation, and the phosphate may still be added to the aqueous material subsequent hydrochloric acid treatment. For example, potassium phosphate may be added, which may be less likely to form precipitates in the aqueous materials, and may be added in lower concentrations to further limit precipitates. Additionally, in some embodiments, one or more organic phosphate compounds may be used instead of any inorganic phosphate compounds, which may be more likely to produce mineral precipitation. As one example, glycerophosphate may not react with water, and thus, precipitation may not occur, although it is to be understood that any other phosphate compounds, including organic or inorganic phosphate may be used in any embodiment encompassed by the present technology.

Temperature within the formation environment, along with materials to be injected, may be controlled to facilitate conversion to methane, as well as viability of the microorganisms. In some embodiments the aqueous material may be delivered within particular temperature and pressure ranges. The aqueous material may be delivered at a relatively increased temperature and pressure, such as above a formation environment temperature, which may increase delivery capability and material incorporation, and may also reduce viscosity of the material provided. For example, the aqueous solution may be delivered to the formation environment at a temperature that is greater than or about 25° C., and in some embodiments the aqueous material may be delivered at a temperature greater than or about 30° C., greater than or about 40° C., greater than or about 50° C., greater than or about 60° C., greater than or about 70° C., greater than or about 80° C., greater than or about 90° C., or higher. In some embodiments the temperature may be maintained less than or about 100° C. to limit a temperature effect within the formation environment, such as which may affect the microorganisms. In some embodiments the temperature may be equal to a downhole temperature within the formation that may be measured prior to delivery of the aqueous material.

The aqueous material may be delivered within the formation environment at a pressure that may facilitate distribution within the formation environment, and may ensure adequate distribution of the glycerol and/or waste product through pores and equipment into the formation. For example, the aqueous material may be delivered at a pressure greater than or about 0.7 MPa, and in some embodiments the delivery pressure of the aqueous material may be greater than or about 1 MPa, greater than or about 2 MPa, greater than or about 3 MPa, greater than or about 5 MPa, greater than or about 7 MPa, greater than or about 9 MPa, greater than or about 10 MPa, greater than or about 12 MPa, greater than or about 14 MPa, greater than or about 15 MPa, greater than or about 16 MPa, greater than or about 17 MPa, greater than or about 18 MPa, greater than or about 20 MPa, or higher.

Additional nutrients and materials may be provided in the aqueous solution in some embodiments of the present technology. Examples of mineral amendments may include the addition of chloride, ammonium, phosphate, sodium, magnesium, potassium, and/or calcium, among other kinds of minerals, in any concentration or combination. Metal amendments may include the addition of manganese, iron, cobalt, zinc, copper, nickel, selenate, tungstenate, and/or molybdate to the isolate, among other kinds of metals. Vitamin amendments may include the addition of pyridoxine, thiamine, riboflavin, calcium pantothenate, thioctic acid, p-aminobenzoic acid, nicotinic acid, vitamin B12, 2-mercaptoehanesulfonic acid, biotin, and/or folic acid, among other vitamins. Yeast extract may be included to provide further nutrients to the microorganisms and may include digests and extracts of commercially available brewers and bakers yeasts. A non-exhaustive list of additional minerals and materials that may be included in any amount or ratio include ammonium chloride, cobalt chloride, copper chloride, manganese sulfate, nickel chloride, nitrilotriacetic acid trisodium salt, potassium monophosphate, potassium diphosphate, sodium molybdate dihydrate, sodium tripolyphosphate, sodium tungstate, zinc sulfate, or some other phosphorus-containing compound, sodium-containing compound, sulfur-containing compound, or carboxylate-containing compounds, such as acetate and formate, for example.

Methanogenic bacteria within the formation environment may facilitate formation of methane. Identifying additional species within the formation environment may also improve digestion of waste glycerol or waste products and constituent materials. For example, microorganisms from the genus Thermotoga may also consume glycerol or carbonaceous materials of waste product streams, and incorporation of this bacteria, or identification of the existence within the formation may be beneficial. Increasing the viability of Thermotoga may be performed by providing additional materials or nutrients that may be selectively consumed by Thermotoga. For example, in some embodiments one or more carbohydrate polymers, such as xylan, may be provided in the aqueous material being delivered. This may further increase the Thermotoga population, which may improve glycerol consumption.

As explained previously, the production of methane within the formation environment may also produce additional materials, such as hydrogen and carbon dioxide. Carbon dioxide that may be produced may be at least partially sequestered within the environment, such as partially in solution with water, or as bicarbonate dissolved in water, and partially into non-mobile oil. However, carbon dioxide that may be released may be recovered with methane produced at the outlet locations. In some embodiments, the present technology may include separating carbon dioxide from the recovered methane. Although the carbon dioxide may be exhausted, in some embodiments the carbon dioxide may be re-injected with subsequent aqueous materials. This may aid in maintaining pressure within the formation, and which may increase movement and production within the environment. Recovered water may be recirculated in any number of ways prior to re-injection in a subsequent treatment. Deleterious materials may be neutralized or removed, when necessary, and then additional glycerol and/or nutrients may be incorporated prior to being re-injected within the environment. The re-injection process may be continuous, or the process may be performed in stages, where the environment may be sealed for a period of time subsequent an injection to allow generation of biogas.

Additionally, carbon dioxide that remains within the formation water or hydrocarbon materials may be converted to bicarbonate. Although through dispersion bicarbonate may be distributed throughout the formation environment, in some embodiments, cycling of aqueous material through the formation may cause bicarbonate concentrations in water as well as hydrocarbon materials, such as oil, to increase over time, which may cause changes to environmental pH and/or microbial activity. Hence, according to some embodiments of the present technology, methods may include reducing a bicarbonate concentration within the aqueous material. As one non-limiting example, as aqueous material is recovered from the formation environment, and prior to re-injection when performed, a bicarbonate concentration may be reduced. For example, calcium carbonate may be generated and recovered as a solid from the aqueous materials, among other bicarbonate removal processes, which may cause a reduction in bicarbonate concentration as processing continues. This may ensure buildup within the formation is controlled, which may limit effects on the microbial environment.

Consequently, gaseous products produced, as well as gaseous products recovered from the in situ environment in embodiments of the present technology, may be characterized by an enriched concentration of methane. Unlike ex situ bioreactors, which may produce and recover relatively equal amounts of methane and carbon dioxide when performing methanogenesis, the present technology may produce gaseous products that may be characterized by an increased percentage of methane relative to other materials. For example, recovered gas products as produced from a well at the in situ environment, and without additional recovery treatments, may be characterized by a methane concentration of the gaseous products of greater than or about 50 vol. %, and may be characterized by a methane concentration of greater than or about 55 vol. %, greater than or about 60 vol. %, greater than or about 65 vol. %, greater than or about 70 vol. %, greater than or about 75 vol. %, greater than or about 80 vol. %, greater than or about 85 vol. %, greater than or about 90 vol. %, greater than or about 95 vol. %, or more, with the residual material being nitrogen, hydrogen, and/or carbon dioxide.

Similarly, recovered gas products as produced from a well at the in situ environment, and without additional recovery treatments, may be characterized by a carbon dioxide concentration of the gaseous products of less than or about 45 vol. %, and may be characterized by a carbon dioxide concentration of less than or about 40 vol. %, less than or about 35 vol. %, less than or about 30 vol. %, less than or about 25 vol. %, less than or about 20 vol. %, less than or about 15 vol. %, less than or about 10 vol. %, less than or about 5 vol. %, less than or about 2 vol. %, less than or about 1 vol. %, or less, and in some embodiments may be substantially or essentially devoid of carbon dioxide. This may provide a renewable bioreactor, which may utilize waste carbonaceous source material to produce methane, and which may control or limit greenhouse gas production.

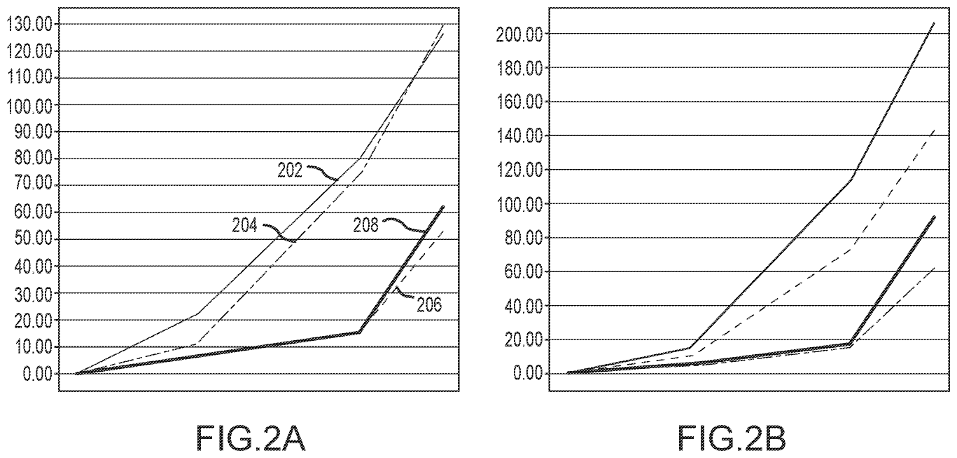

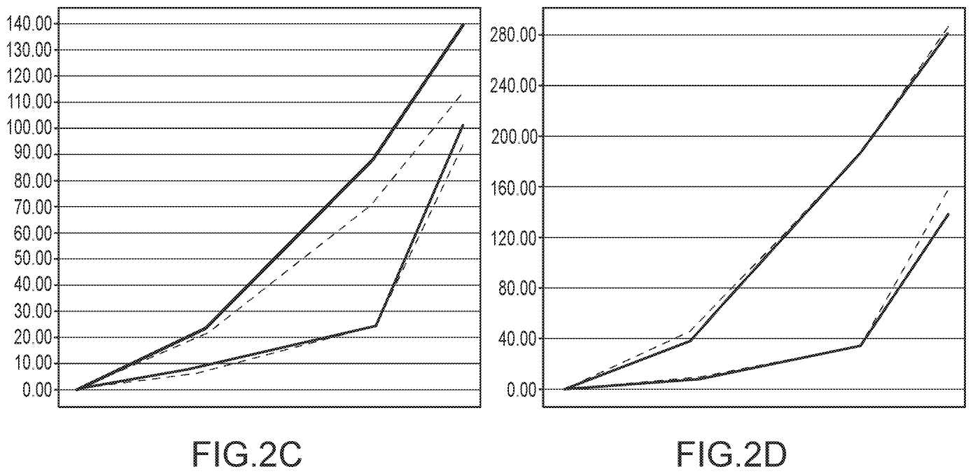

FIGS. 2A-2D illustrate the effects of glycerol at a first concentration and/or additional nutrients and material incorporation on a microorganism community for producing methane. Methane production is illustrated along the Y-axis in each graph, and shown in units of micromoles. FIG. 2A illustrates the production based on only incorporation of glycerol. FIG. 2B illustrates the further incorporation with glycerol of materials including phosphate and ammonium chloride. FIG. 2C illustrates the incorporation of glycerol as well as materials including phosphate and yeast extract. FIG. 2D illustrates the incorporation of glycerol as well as materials including phosphate, acetate, and yeast extract. As shown, incorporation of additional nutrients increases the methane production. Additionally, samples were tested in each case with and without the incorporation of an additional hydrocarbon material. As shown in FIG. 2A, lines 202 and 204 show the results of tests performed with additional incorporation of carbonaceous material, while lines 206 and 208 show the results of tests performed without additional incorporation of carbonaceous material. In all four examples shown in FIGS. 2A-2D, production was increased in the presence of additional hydrocarbon material, as shown by the top two lines in each graph. Hence, by performing the process in an in situ bioreactor, such as in a formation environment including additional hydrocarbon material, the methane produced from consumption of glycerol may be increased beyond what may be produced in an ex situ environment. The present technology may therefore provide processes for efficiently and economically consuming waste glycerol to produce biogas.

As discussed previously, the present technology may utilize multiple waste product and/or carbonaceous material streams to further increase methane production over individual waste product streams, according to some embodiments of the present technology. Although any number of individual waste product streams may be used, either adjusted or unadjusted by any configuration material or operation discussed previously, in some embodiments complementary waste product streams may be combined in one or more ways and delivered to the formation. Complementary streams may include any number of characteristics that may provide a more beneficial combination of materials to the consortium, which may synergistically improve methane production, and in some embodiments may boost methane production higher than either individual stream used alone. Combinations of any two or more waste product streams discussed above may be used, and any of the streams may be amended, adjusted, or otherwise configured by any process or material discussed previously. The inventors have identified multiple complementary waste product sources that when combined may improve methane production beyond that of either individual stream, and the examples below are not intended to limit the present technology or claims in any way.

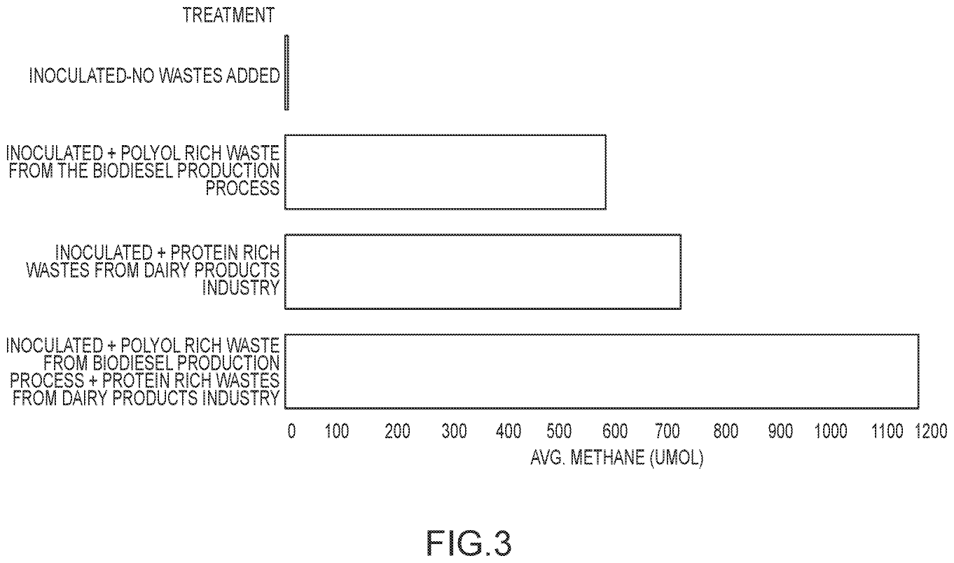

FIG. 3 is a chart illustrating the synergistic effects of combining supportive waste product streams according to some embodiments of the present technology. The chart shows results of a microcosm study that was conducted to evaluate the synergistic role of combining multiple types of configurable support material, such as waste product or carbonaceous materials, for the in situ creation of methane in oil fields. Produced fluids from a domestic oil field were collected anaerobically and used in the experiment. Eight 125 mL (164.4 mL volume) serum bottles were anaerobically filled to zero headspace from the produced fluids. 20 mL of UHP nitrogen was added to each bottle while venting to set an N2 headspace, 0 psi at ambient temperature. All serum bottles were inoculated with microorganisms derived from the same oil field and previously enriched on waste product carbonaceous material derived from a biodiesel production process.

Six of the eight serum bottles had additional waste product carbonaceous materials added. Two were amended with configurable support material derived from the biodiesel production process, two were amended with configurable support material derived from dairy waste, and two were amended with a combination of configurable support material derived both from the biodiesel production process and from dairy waste. Two serum bottles remained free of additional configurable support material as controls. In the experiments conducted the dominant organic material from the biodiesel waste stream was polyol compounds, and the dominant organic material from the dairy products waste stream was protein-rich compounds. The total amount of configurable support material added under each test condition was identical on a chemical oxygen demand (COD) basis. Where a single configurable support material was added, the final concentration was 100 mg/L COD. Where the combination of two configurable support materials was added, each was added at 50 mg/L COD for a total of 100 mg/L COD. Bottles were then stored at 55° C. to mimic the in situ temperature.

After one week of incubation, headspace gas was analyzed for methane creation from the configurable support materials by gas chromatography. FIG. 3 illustrates the average methane creation of the duplicate test microcosms at seven days after experimental setup. Data in FIG. 3 shows that adding multiple configurable support materials together provides a synergistic effect in enhancing methane creation. Providing both configurable support materials together increased the yield of methane by 49% or 37%, relative to adding just a single material derived from the biodiesel production process or from dairy waste, respectively.

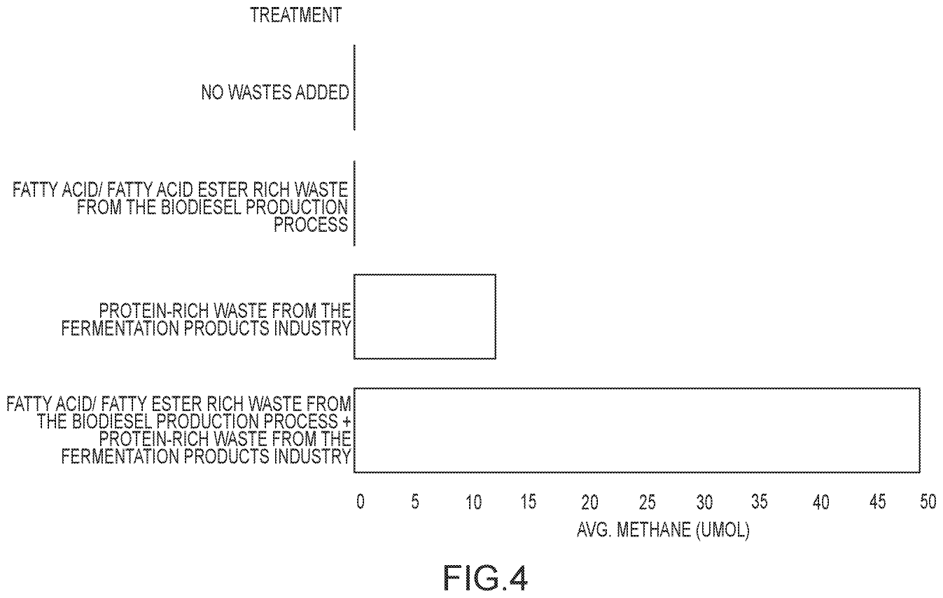

FIG. 4 is a chart illustrating the synergistic effects of combining supportive waste product streams according to some embodiments of the present technology, and may illustrate an additional exemplary combination of waste product stream materials that may produce complementary effects from the consortium. The figure illustrates an example of the synergistic effects of combining supportive waste product streams according to some embodiments of the present technology for which no inoculum may be added. The chart shows results of an additional microcosm study that was conducted to evaluate the effect of combining multiple types of configurable support material, such as waste products or carbonaceous materials, for the in situ creation of methane in oil fields. Produced fluids from a domestic oil field were collected anaerobically and used in the experiment. Eight 30 mL (37 mL volume) serum bottles were anaerobically filled to zero headspace from the produced fluids. 10 mL of UHP nitrogen was added to each bottle while venting to set an N2 headspace of 0 psi at ambient temperature.

Six of the eight serum bottles had additional waste product carbonaceous materials added. Two were amended with configurable support material derived from the biodiesel production process, two were amended with configurable support material derived from the fermentation products industry, and two were amended with a combination of configurable support material derived both from the biodiesel production process and from fermentation products industry waste. Two serum bottles remained free of additional configurable support material as controls. Different from the previous experiments discussed above, in these experiments conducted the dominant organic material from the biodiesel waste stream was fatty acid/fatty acid ester compounds, and the dominant organic material from the fermentation products waste stream was protein-rich compounds. The total amount of configurable support material added under each test condition was identical on a chemical oxygen demand (COD) basis. Where a single configurable support material was added, the final concentration was 500 mg/L COD. Where the combination of two configurable support materials was added, each was added at 250 mg/L COD for a total of 500 mg/L COD. Bottles were then stored at 55° C. to mimic the in situ temperature.

After two weeks of incubation, headspace gas was analyzed for methane creation from the configurable support materials by gas chromatography. FIG. 4 illustrates the average methane creation of the duplicate test microcosms at fourteen days after experimental setup. Data in FIG. 4 shows that adding multiple configurable support materials together provides a synergistic effect in enhancing methane creation. Providing both configurable support materials together increased the yield of methane by 299% or greater, relative to adding a single material derived from either the biodiesel production process or from fermentation waste. As noted above, the results with regard to FIG. 3 are based on inoculated formation water sources, while the results with regard to FIG. 4 are based on non-inoculated formation water sources. This may account for the illustrated differences in methane production. Relative to the results discussed with regard to FIG. 3, the results shown in FIG. 4 illustrate that a proportional outcome of increased methane production from delivery of certain mixed waste streams may occur even for slower or less-responding oil wells.

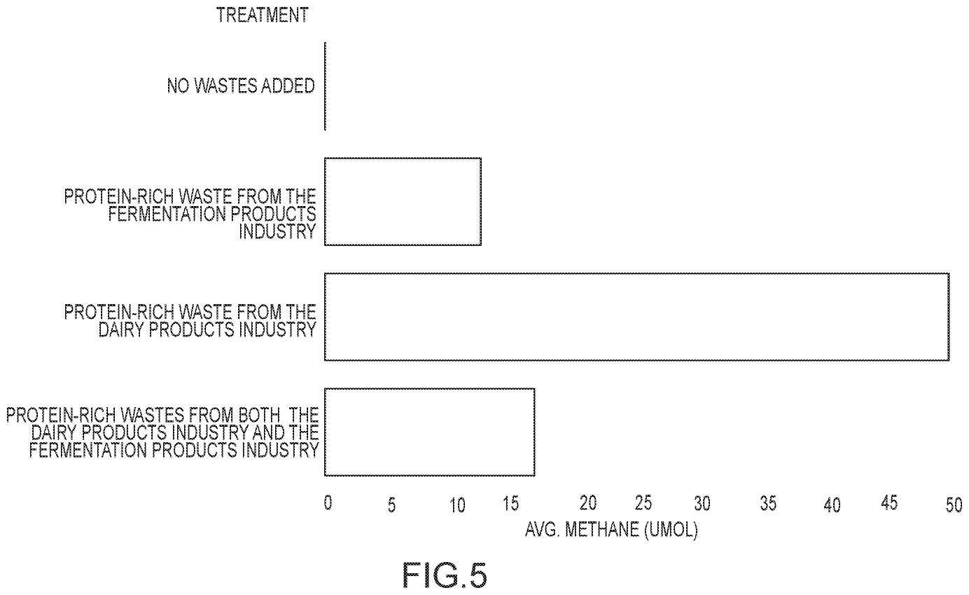

FIGS. 3 and 4 demonstrate the synergistic impacts of adding different types or classes of wastes together to form a complementary, complete nutritional environment for microorganisms. Conversely, adding multiple types of wastes containing related classes of compounds may elicit an antagonistic effect in some circumstances. FIG. 5 is a chart illustrating antagonistic effects of combining multiple waste product streams according to some embodiments of the present technology. The experiment illustrated was constructed and analyzed identically to that of FIG. 4. Eight serum bottles were utilized in the experiment. Six of the eight serum bottles had additional waste product carbonaceous materials added. Two were amended with configurable support material derived from the fermentation products industry, two were amended with configurable support material derived from the dairy products industry, and two were amended with a combination of configurable support material derived both from the fermentation products industry and from the dairy products industry. Two serum bottles remained free of additional configurable support material as controls. The total amount of configurable support material added under each test condition was identical on a chemical oxygen demand (COD) basis. Where a single configurable support material was added, the final concentration was 500 mg/L COD. Where the combination of two configurable support materials was added, each was added at 250 mg/L COD for a total of 500 mg/L COD. Bottles were then stored at 55° C. to mimic the in situ temperature.

After two weeks of incubation, headspace gas was analyzed for methane creation from the configurable support materials by gas chromatography. FIG. 5 illustrates the average methane creation of the duplicate test microcosms at fourteen days after experimental setup. Data in FIG. 5 shows that adding multiple configurable support materials of the same type may have an antagonistic impact on methane creation. Different from the previous experiments discussed above, in these experiments conducted the dominant organic material from the fermentation waste stream was protein-rich compounds, and the dominant organic material from the dairy products waste stream was also protein-rich compounds. Providing both configurable support materials together reduced the yield of methane in one case by 66% and produced similar results to adding just the single configurable support material in the other case. Without being bound by any particular theory, the over-incorporation of protein-rich waste streams may at least partially overwhelm the microorganisms leading to toxicity within the environment. This may reduce the effect leading to detrimental production of methane. Consequently, by balancing the nutritive sources of the waste streams delivered to the formation environment according to some embodiments of the present technology, microorganism consortiums may be bolstered to further improve methanogenic output relative to single waste stream delivery.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having disclosed several embodiments, it will be recognized by those of skill in the art that various modifications, alternative constructions, and equivalents may be used without departing from the spirit of the embodiments. Additionally, a number of well-known processes and elements have not been described in order to avoid unnecessarily obscuring the present technology. Accordingly, the above description should not be taken as limiting the scope of the technology.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

As used herein and in the appended claims, the singular forms “a”, “an”, and “the” include plural references unless the context clearly dictates otherwise. Thus, for example, reference to “a material” includes a plurality of such materials, and reference to “the nutrient” includes reference to one or more nutrients and equivalents thereof known to those skilled in the art, and so forth.

Also, the words “comprise(s)”, “comprising”, “contain(s)”, “containing”, “include(s)”, and “including”, when used in this specification and in the following claims, are intended to specify the presence of stated features, integers, components, or operations, but they do not preclude the presence or addition of one or more other features, integers, components, operations, acts, or groups.

What is claimed is:

1. A method of producing methane in a reservoir, the method comprising:
accessing a consortium of microorganisms in a geologic formation;
delivering an aqueous solution including waste products to the consortium of microorganisms;
increasing production of gaseous materials by the consortium of microorganisms; and
recovering gaseous products from the reservoir, wherein the gaseous products comprise an enriched methane concentration.

2. The method of producing methane in a reservoir of claim 1, wherein the aqueous solution comprises less than or about 50 vol. % waste product, and wherein the aqueous solution comprises produced water extracted from the geologic formation.

3. The method of producing methane in a reservoir of claim 1, wherein the gaseous products recovered from the reservoir are characterized by a concentration of carbon dioxide of less than or about 10 vol. %.

4. The method of producing methane in a reservoir of claim 1, further comprising:
characterizing the environment of the geologic formation, wherein characterizing the environment of the geologic formation comprises identifying, within the geologic formation environment, one or more of a sulfate concentration, a salinity, a temperature, or a pH.

5. The method of producing methane in a reservoir claim 1, wherein the aqueous solution comprises greater than or about 0.1 vol. % glycerol.

6. The method of producing methane in a reservoir of claim 1, further comprising:
recovering effluent aqueous material at an outlet from the reservoir; and
monitoring a concentration of carbonaceous waste within the effluent aqueous material.

7. The method of producing methane in a reservoir claim 1, wherein the aqueous solution delivered is characterized by a pH of less than or about 8.

8. The method of producing methane in a reservoir of claim 7, wherein the pH of the aqueous solution is adjusted by incorporating hydrochloric acid or phosphoric acid.

9. The method of producing methane in a reservoir of claim 8, further comprising:
measuring a calcium concentration of an effluent aqueous material recovered from the geologic formation; and
reducing the pH of the aqueous solution with hydrochloric acid when the calcium concentration is measured to be greater than or about 20 mg/L, or
reducing the pH of the aqueous solution with phosphoric acid when the calcium concentration is measured to be less than or about 80 mg/L.

10. The method of producing methane in a reservoir of claim 9, further comprising:
when the pH of the aqueous solution is reduced with hydrochloric acid, providing a phosphate compound to the aqueous solution.

11. The method of producing methane in a reservoir of claim 1, wherein the aqueous solution delivered into the geologic formation is characterized by a first concentration of salinity and a first concentration of free fatty acids.

12. The method of producing methane in a reservoir of claim 11, wherein a feedstock of the waste product is adjusted over time to increase carbonaceous material in the aqueous solution, and wherein the aqueous solution is adjusted over time to increase free fatty acids to a second concentration greater than the first concentration of free fatty acids.

13. The method of producing methane in a reservoir of claim 1, wherein the aqueous solution further comprises one or more of yeast extract, inorganic nitrogen, a carboxylate material, a metalloid, a vitamin, a mineral, or a metal material.

14. A method of producing methane in a reservoir, the method comprising:
accessing a consortium of microorganisms in a geologic formation;
delivering an aqueous solution incorporating a waste product stream having a carbonaceous waste product at a concentration of greater than or about 10 wt. % to the consortium of microorganisms;
increasing production of gaseous materials from consumption of the waste product by the consortium of microorganisms; and
recovering gaseous products from the reservoir, wherein the gaseous products are characterized by a concentration of carbon dioxide of less than or about 40 vol. %.

15. The method of producing methane in a reservoir of claim 14, further comprising:
identifying microorganisms from genus Thermotoga.

16. The method of producing methane in a reservoir of claim 15, further comprising:
incorporating xylan in the aqueous solution delivered to the consortium of microorganisms.

17. The method of producing methane in a reservoir of claim 14, further comprising:
separating the carbon dioxide from methane recovered from the reservoir.

18. The method of producing methane in a reservoir of claim 17, further comprising:
re-injecting the carbon dioxide separated from the methane into the geologic formation.

19. A method of producing methane in a reservoir, the method comprising:
accessing a consortium of microorganisms in a geologic formation;
delivering an aqueous solution incorporating waste product having a glycerol concentration of greater than or about 0.1 vol. % to the consortium of microorganisms;
increasing production of gaseous materials from consumption of the waste product by the consortium of microorganisms; and
recovering gaseous products from the reservoir, wherein the gaseous products are characterized by a concentration of methane of greater than or about 51 vol. %.

20. The method of producing methane in a reservoir of claim 19, further comprising:
characterizing one or more aspects of the geologic formation selected from a group including temperature, salinity, sulfate concentration, alkalinity, pH, or permeability; and
modifying the one or more aspects of the geologic formation.
